(19) United States
(12) Patent Application Publication
(10) Pub. No.: US 2019/0142330 A1
(43) Pub. Date: May 16, 2019

(54) Bra-Type Breast Tumor Self-Detection Mask

(71) Applicant: Fu-Chung Ko, Taichung City (TW)

(72) Inventor: Fu-Chung Ko, Taichung City (TW)

(21) Appl. No.: 15/811,428

(22) Filed: Nov. 13, 2017

(51) Int. Cl. A61B 5/00; A61B 8/08; A41C 3/00

(52) U.S. Cl. CPC A61B 5/4312; A61B 8/0825; A61B 2560/045; A61B 5/6804; A41C 3/0064

(57) ABSTRACT

A bra-type breast tumor self-detection mask includes a double-layered structure formed by a top surface and a bottom surface. Edges on the top surface and the bottom surface are sealed, a small space is formed between the top surface and the bottom surface, and the small space is filled with a lubrication element. The self-detection mask fully covers breasts in a large area just like a bra. The self-detection mask is provided with positioning elements, a face on the top surface or the bottom surface is provided with plural recording lines which surround into plural regions on which are marks. The top surface provides for the pressing and the horizontal movement of the fingers, and the bottom surface touches the breast skin for one-time detection. The breast tumor can be touched and recorded accurately without moving the self-detection mask.

### FIELD OF THE INVENTION

The disclosure relates to medicine and more particularly to disease detection technologies.

### BACKGROUND

It is known that for most of the breast cancers in an early stage, there is no symptom and ache. For a fat breast or a lump deep in the breast, the tumor is not easy to be touched. In addition, for the stage 0 or 1 breast cancer, the tumor is unable to be touched by self-detection alone. According to Taiwan Clinical Oncology Research Foundation, a simple method of practicing palpation is to put two fingers on the back of another hand, and then let the fingertips to slide on the back of hand slowly, without moving the skin. In the meantime, one should feel the protruded fasciae or bones underneath the skin, and then gently press down the fingers, allowing the fingertips to move on the structures underneath the skin and turning the fingers in small circles from one point toward another direction, until covering the whole back of hand. Using this method to move on the skin, one can know whether there is a tumor underneath the skin for early detection and early treatment. However, as not all women are professional nursing personnel, it is unable to know whether there is a tumor by touching with the fingers alone. Especially when the tumor is small or in early development, the pressure acted by a finger onto a single point on the skin will result in the displacement or shrinkage of the tumor, so that the tumor cannot be found. As it requires a large pressure to a single point on the skin by the finger, the friction force is large too. Therefore, upon moving horizontally on the skin, the fingers will still displace horizontally in a small area around a single point and do not displace actually. Accordingly, the tumor in the breast cannot be touched actually for a full breast or when a lump is deep in the breast, and the self-detection will normally find no anomaly, thereby missing the chance of early detection for early treatment.

A previous patent application, “Single-Handed Detection Sag for a Breast Lump,” includes a bag of an adequate amount of oil and a glove. The bag is made of plastic with high elasticity and is formed by sealing two layers of plastic tightly. A side of the sealing edge is reserved with a non-sealed injection opening to fill the oil. The glove is disposed on a surface of the bag and the shape of the bag fits with the bag. A wear opening which is slightly concaved is disposed on the glove opposite to the injection opening of the bag. In addition to the wear opening, the fitting edge between the bag and the glove is processed into a tightly combined bonding edge by a high-frequency wave or an ultrasonic wave. An adequate amount of oil is filled into the bag from the injection opening, and then the injection opening is sealed, forming a single-handed detection sag for a breast lump, so that a single hand can be sheathed into the glove to conduct the detection operation. After sheathing a single hand into the glove, the bag is made to cover a breast, and fingers of a single hand move back and forth on an outer surface of the bag to detect whether there is a lump. Furthermore, after covering the breast with the bag, the friction force resulting from moving the fingers can be decreased by the oil in the bag, and the sensibility of fingers in perceiving the protruded lump can be improved by the amplification function of the double-layered bag and the oil inside the bag, thereby achieving the effect of facilitating the early detection of lump for early treatment.

That invention includes primarily a bag and a glove that are formed by two layers of plastic with three surfaces. The two layers are the bag and the glove, an upper surface and a middle surface in the three surfaces form the glove, and the middle surface and a bottom surface in the three surfaces form the bag. The bag is filled with an adequate amount of oil, and the injection opening is then sealed. A hand is sheathed into the glove, with fingers pressing the middle surface and the bottom surface. As the oil is disposed between the middle surface and the bottom surface, the bottom surface will touch the breast skin. Although the pressure is large, the friction force between the middle surface and the bottom surface is small. Therefore, the fingers are easy to slide between the middle surface and the bottom surface to slide easily on the breast skin and press the breast skin deeply, so that the tumor in the breast can be touched and identified actually, thereby achieving the effect of early detection for early treatment.

However, that invention is a single bag and a single glove. One of the shortcomings is that the range of horizontal movement after the fingers are sheathed into the glove is limited. Therefore, the glove should be raised up constantly to move again. As the glove is raised up constantly, the tumor that was touched originally cannot be recorded on the glove for a next examination to check whether the tumor is enlarged, and thus a new detection should be taken. This is a second shortcoming. As a single bag and a single glove are manufactured for a single breast, it is not easy to cover a full breast. In addition, due to the limitation in area, it is very difficult for the fingers to move quickly to touch for detection, which wastes the time in detection. This is a third shortcoming. After the fingers have been sheathed into the glove, as the glove itself is limited in the vertical space of movement, the fingers will be bulged on the upper surface of the glove by the effect of the upper surface and the inner space of the glove, along with the middle surface and the bottom surface of the glove, to affect the sense of touch. This is a fourth shortcoming. The action force for attaching and positioning the glove and the bag on the breast depends upon the force of palm and fingers. The fingers do pressing, and on the other hand, the palm will need to act a force on the glove and the bag to attach on the breast. Therefore, the glove and the bag will slide off frequently. This is a fifth shortcoming.

### SUMMARY OF THE INVENTION

Disclosed herein is a bra-type breast tumor self-detection mask, and more particularly to a bra-type breast tumor self-detection mask which is constituted by a double-layered structure and a lubrication element, with the double-layered structure including a top surface and a bottom surface. The bra-type breast tumor self-detection mask covers breasts in a large area, just like a bra attached and positioned on the breasts. The self-detection mask is not limited by a glove layer, and fingers can move autonomously on the top surface, without affecting the autonomous movement of the palm and the fingers, as well as the magnitude of pressure acted by the fingers. When a tumor is touched at the first time, it is recorded according to a mark on its belonged region, acting as a base of determination for a next examination to check whether the tumor is enlarged, without requiring a new detection.

The conventional device is a single bag and a single glove. Although it facilitates self-detection to check whether there is a breast tumor, the sense of touch is affected due to the limitation in horizontal movement and vertical space of movement of the glove. In addition, as the glove needs to be raised up constantly to move again, and the result cannot be recorded on the glove, it will waste a lot of time in detection. These shortcomings troubled vendors and users.

Accordingly, disclosed herein is a bra-type breast tumor self-detection mask. The mask includes a double-layered structure formed by a top surface and a bottom surface. Edges on the top surface and the bottom surface are sealed. A small space is formed between the top surface and the bottom surface and can be filled with a lubrication element. In some embodiments, a bra-type breast tumor self-detection mask is constituted by a single top surface and a single bottom surface to fully cover the breasts in a large area. The self-detection mask is also provided with positioning elements, allowing the self-detection mask to be attached and positioned on the breasts. The top surface provides for pressing and horizontal movement of the fingers, whereas the bottom surface touches the breast skin.

A first object is to provide a bra-type breast tumor self-detection mask. The friction force between the top surface and the bottom surface is low, and the bottom surface touches the breasts. When being subjected to a force, the top surface will be shifted with respect to the bottom surface, and the fingers can actually move horizontally by touching the top surface, so that a breast tumor can be touched and found out.

A second object is to provide a bra-type breast tumor self-detection mask. The self-detection mask is not limited by a glove layer that the fingers can move completely on the top surface autonomously, without affecting the pressure acted by the fingers.

A third object is to provide a bra-type breast tumor self-detection mask. The self-detection mask can cover the breasts in a large area, even beneath armpits, which fully covers the area where the tumors may grow.

A fourth object is to provide a bra-type breast tumor self-detection mask. The self-detection mask is attached stably on the breasts without using a palm to act a force onto the self-detection mask to attach on the breasts. The self-detection mask will not slide off and the fingers can focus on pressing the breast skin.

A fifth object is to provide a bra-type breast tumor self-detection mask. With the self-detection mask, the glove will not need to be raised up to move again, and the region of the tumor that was touched originally can be marked and recorded, acting as a base of determination for a next examination to check whether the tumor is enlarged, without requiring a new detection.

A sixth object is to provide a bra-type breast tumor self-detection mask. The self-detection mask can easily cover a full breast without limitation in area. The fingers can act a force easily on the self-detection mask to move horizontally in a fast speed to touch and detect, which shortens the time in detection.

A seventh object is to provide a bra-type breast tumor self-detection mask. For the self-detection mask, only the top surface and the bottom surface are subjected to a force with each other to shift with respect to each other, which avoids the shortcoming in a prior art that after the fingers are sheathed into the glove layer, the fingers will be bulged on the upper surface of the glove layer, along with the middle surface and the bottom surface, to cause the loss in the sense of touch.

To enable a further understanding of the said objectives and the technological methods herein, the brief description of the drawings below is followed by the detailed description of the preferred embodiments.

### BRIEF DESCRIPTION OF THE DRAWINGS

FIG. 1 shows a planar view of the mask.

FIG. 2 shows an enlarged planar view of a primary part of the mask.

FIG. 3 shows a cutaway view of the mask.

FIG. 4 shows a cutaway view of fingers pressing a top surface of the mask.

FIG. 5 shows a planar view that the mask is applied to breasts.

### DETAILED DESCRIPTION OF THE PREFERRED EMBODIMENTS

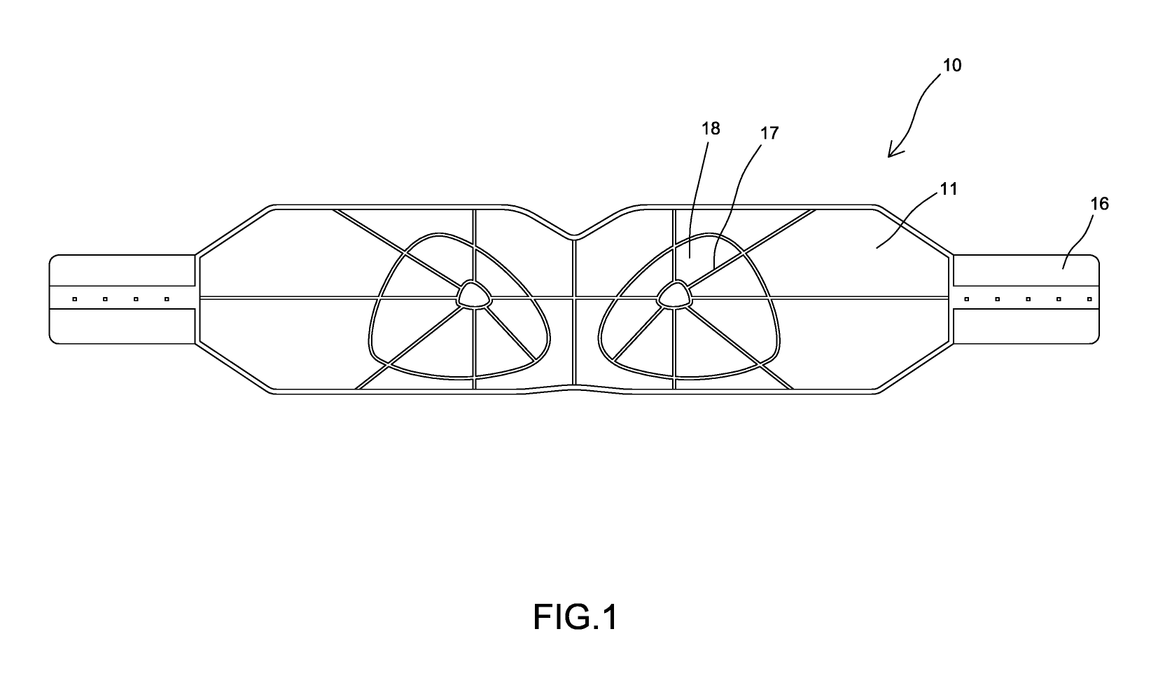

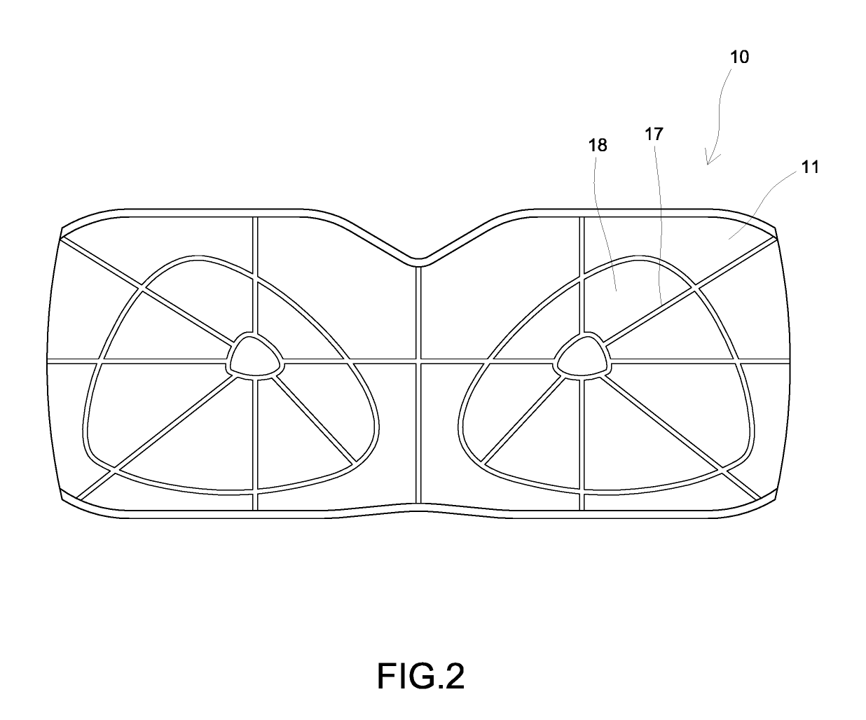

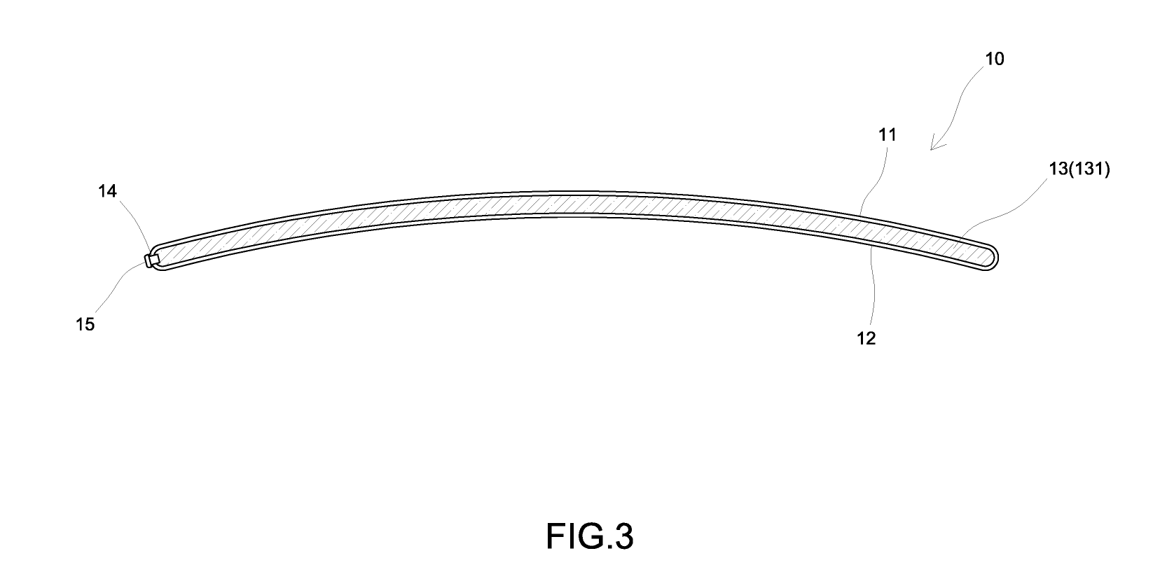

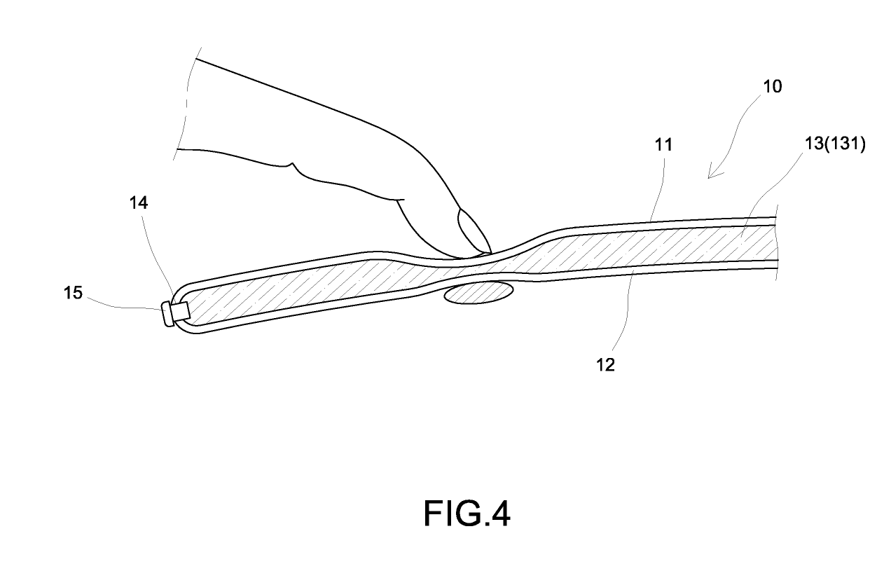

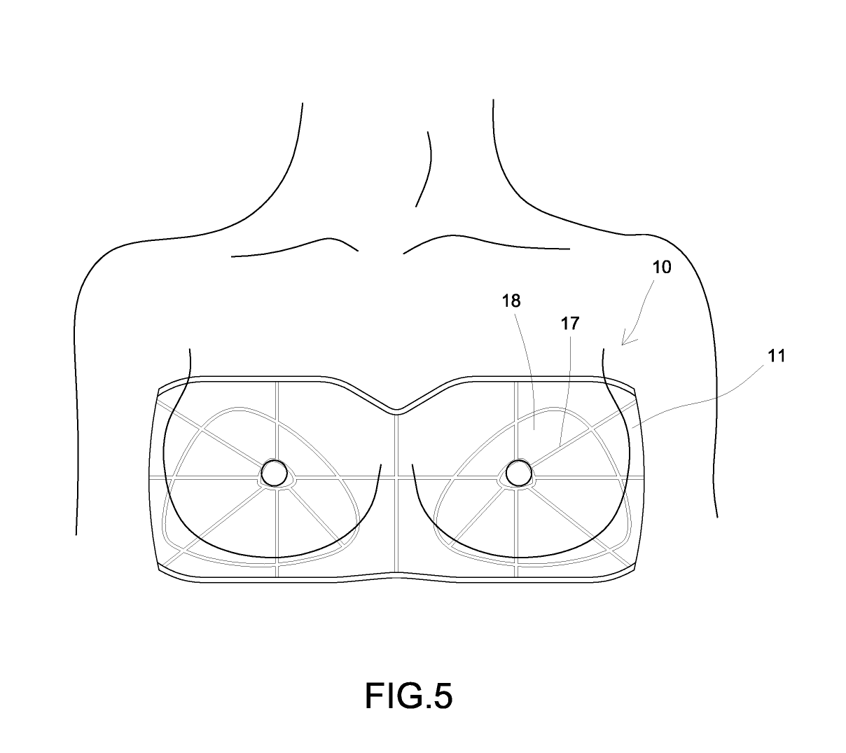

Referring to FIGS. 1 to 5, a bra-type breast tumor self-detection mask is disclosed. The self-detection mask 10 is a rectangular double-layered structure in a shape of bra and is attached on breasts just like the size of a bra. The self-detection mask 10 comprises a top surface 11, a bottom surface 12 and a small space 13. The top surface 11 and the bottom surface 12 are all made of a soft and impermeable material, with a hole 14 in a pre-determined location on the top surface 11 or the bottom surface 12. The hole 14 is installed with a plug 15. Edges on the top surface 11 and the bottom surface 12 are sealed, forming a small space 13 between the top surface 11 and the bottom surface 12. The self-detection mask 10 is integral in a large area, and is able to cover breasts and expand to underarms, not only a breast at one side.

An interior of the small space 13 is provided with a lubrication element 131 which can be a liquid element, such as water, oil or talcum powder, to decrease the friction force between the top surface 11 and the bottom surface 12. The plug 15 is opened, the lubrication element 131 is filled into the small space 13 from the hole 14, and the hole 14 is sealed by the plug 15 (or the edges on the top surface 11 and the bottom surface 12 are sealed directly by a high-frequency wave). Two sides of the top surface 11 or the bottom surface 12 can all provide for the pressing and horizontal movement of the fingers. The large area of the top surface 11 or the bottom surface 12 can all be used to touch the breast skin. Two sides of the top surface 11 or the bottom surface 12 can be provided respectively with a positioning element 16 which is a brace, like a brace of bra that is snapped on a back of a person, allowing the self-detection mask 10 to be positioned on the breasts. On the other hand, the edges on the bottom surface 12 can be provided with adhesive points (not shown in the drawings) which are adhesive silica gel. The adhesive points are attached on peripheries of the breasts, allowing the self-detection mask 10 to be attached stably on the breasts. A face on the top surface 11 or the bottom surface 12 is provided with plural recording lines 17 which surround into plural regions 18. The device is provided with plural large and small regions 18 on which are marks (not shown in the drawings), to facilitate recording where the tumors are for each self-detection.

Accordingly, in some embodiments, upon using the bra-type breast tumor self-detection mask, the entire self-detection mask 10 is first attached on the breasts, and the positioning elements 16 at two sides are snapped on the back of a person, allowing the self-detection mask 10 to be stably attached on the breasts in a large range and large area. The fingers touch the top surface 11 of the self-detection mask 10 directly, and the bottom surface 12 touches the breast skin. In addition to acting a force, the fingers also move horizontally in circles. As the friction force between the top surface 11 and the bottom surface 12 is low, the top surface 11 and the bottom surface 12 will not be attached together, but are shifted with respect to each other by being subjected to the force with respect to each other. The fingers can actually move in horizontal direction to touch and find out a breast tumor by touching the top surface 11.

In certain embodiments, there is no conventional glove layer and there are no problems resulting from the fingers being sheathed into the glove layer. Therefore, the mask is not limited by the space of the glove layer, the vertical space and the horizontal space of movement, and can be used to press autonomously without any obstacle whether in moving horizontally or pressing from top to bottom. There is no need to move the self-detection mask 10 for detection, and the fingers can feel the top surface 11 and the bottom surface 12 accurately to detect the breast tumor.

In certain embodiments, the disclosed mask has the following features:
1. The self-detection mask 10 is not limited by the glove layer. When the fingers move autonomously on the top surface 11, the autonomous movement of the palm and the fingers as well as the magnitude of pressure acted by the fingers will not be affected.
2. The self-detection mask 10 acts as a bra to cover the breasts in a large area, even to underarms, which totally covers the area where the tumors may grow.
3. The self-detection mask 10 is provided with the positioning elements 16 that are snapped on the back of a person. Therefore, the self-detection mask 10 can be attached on the breasts without using the palm to act a force on the self-detection mask 10 to attach on the breasts. The self-detection mask 10 will not slide off and the fingers can focus on pressing the breast skin.
4. It no longer needs to raise the glove layer to move again.
5. The tumor that was touched originally can be recorded on the region 18 with the marks, acting as a base of determination for a next examination to check whether the tumor is enlarged, without requiring a new detection.
6. The self-detection mask is easy to cover a full breast, and the fingers can easily act a force on the self-detection mask to move and touch for detection in a fast speed, which shortens the time in detection.
7. When using the fingers to press, only the top surface 11 and the bottom surface 12 are subjected to a force with respect to each other and are thus shifted with respect to each other, which avoids the shortcoming in a prior art that after the fingers are sheathed into the glove layer, the fingers will be bulged on the upper surface of the glove layer, along with the middle surface and the bottom surface, to cause the loss in the sense of touch.

It is of course to be understood that the embodiments described herein is merely illustrative of the principles of the disclosure and that a wide variety of modifications thereto may be effected by persons skilled in the art without departing from the spirit and scope of the disclosure as set forth in the following claims.

What is claimed is:

1. A bra-type breast tumor self-detection mask, comprising a double-layered structure formed by a top surface and a bottom surface, wherein edges on the top surface and the bottom surface are sealed, a small space is formed between the top surface and the bottom surface, and the small space is filled with a lubrication element, with that the bra-type self-detection mask constituted by a single top surface and a single bottom surface fully covers breasts in a large area, positioning elements are disposed on pre-determined locations on the self-detection mask to attach and position the self-detection mask on the breasts, the lubrication element is a liquid element, a face on the top surface or the bottom surface of the self-detection mask is provided with plural recording lines which surround into plural regions on which are marks, the top surface provides for the pressing and the horizontal movement of the fingers without limitation in space, the bottom surface touches the breast skin for one-time detection, and the breast tumor is touched accurately without moving the self-detection mask.

2. The bra-type breast tumor self-detection mask according to claim 1, wherein a pre-determined location on the top surface or the bottom surface is provided with a hole which is installed with a plug.

3. The bra-type breast tumor self-detection mask according to claim 1, wherein two sides of a positioning element on the top surface or the bottom surface are provided respectively with a brace, whereby the two braces being snapped together.
